(19) United States
(12) Patent Application Publication
(10) Pub. No.: US 2026/0007585 A1
(43) Pub. Date: Jan. 8, 2026

(54) Oral Care Compositions Comprising Oxalic Acid

(71) Applicant: The Procter & Gamble Company, Cincinnati, OH (US)

(72) Inventors: Phillip Asa Drake, Mason, OH (US); Andrew Frederic Groth, Mason, OH (US); Tiffany Celeste Hare, West Chester, OH (US); Paul Albert Sagel, Maineville, OH (US); Rachel Anne Trenner, Centerville, OH (US)

(21) Appl. No.: 19/327,214

(22) Filed: Sep. 12, 2025

Related U.S. Application Data
(63) Continuation of application No. 17/580,688, filed on Jan. 21, 2022.
(60) Provisional application No. 63/143,970, filed on Feb. 1, 2021.

(51) Int. Cl. A61K 8/362; A61K 8/73; A61Q 11/00

(52) U.S. Cl. CPC A61K 8/362; A61K 8/731; A61Q 11/00; A61K 2800/28; A61K 2800/30

(57) ABSTRACT

Oral care compositions comprising oxalic acid and less than 1%, by weight of the oral care composition, of cellulose derivative. Oral care compositions comprising oxalic acid and less than 1%, by weight of the oral care composition, of carboxymethylcellulose. Oral care compositions comprising oxalic acid and free of carboxymethylcellulose.

### FIELD OF THE INVENTION

The present invention relates to oral care compositions comprising oxalic acid and less than 1%, by weight of the oral care composition, of cellulose derivative. The present invention relates to oral care compositions comprising oxalic acid and less than 1%, by weight of the oral care composition, of carboxymethylcellulose. The present invention relates to oral care compositions comprising oxalic acid and substantially free of, essentially free of, or free of carboxymethylcellulose.

### BACKGROUND OF THE INVENTION

The four major components of human teeth are enamel, cementum, pulp, and dentin. The dentin layer of teeth includes microscopic channels, known as dentinal tubules, which run from the pulp to the exterior cementum layer (below the gum line) or the exterior enamel layer (above the gum line).

The dentin layer, which is naturally protected and sealed by enamel or cementum, can become exposed through gum recession, enamel wear, and/or erosion. Once the dentin layer is exposed, variations in temperature, tactile sensations, and/or osmotic insults can cause a rapid pulse of fluid through the exposed dentin tubules. The rapid pulse of fluid stimulates the pulpal nerve and leads to transient pain in the oral cavity, which is commonly referred to as tooth sensitivity.

Tooth sensitivity can be treated by creating a chemical barrier over the exposed dentin and/or filling the dentin tubules with a solid material. Stannous ions can be added to oral care compositions to provide a tooth sensitivity relief by depositing stannous ions on the surface of the dentin and/or enamel. However, the tooth sensitivity relief provided by stannous ions is only temporary as stannous ions only remain on the tooth's surface for hours.

Longer lasting tooth sensitivity can be achieved by precipitating and/or crystalizing solid material within the dentin tubules. Crystallization of material into dentin tubules can be preferred because crystalized material can be more resistant to subsequent solubilization after deposition within the dentin tubules. However, certain common components of oral care compositions can interfere with the crystallization of materials into dentin tubules.

Thus, there is a need for oral care compositions that do not inhibit the crystallization of material, such as oxalate salts, into dentin tubules.

### SUMMARY OF THE INVENTION

Disclosed herein are oral care compositions that can crystalize material into dentin tubules. The disclosed oral care compositions can lead to crystalized material in exposed dentin tubules in as little as a single oral care treatment application, and can lead to more extensive deposition of crystalized material following multiple usage cycles.

Disclosed herein is an oral care composition comprising (a) oxalic acid; and (b) less than about 1%, by weight of the oral care composition of cellulose derivative.

Disclosed herein is an oral care composition comprising (a) oxalic acid; and (b) less than about 1%, by weight of the oral care composition of carboxymethylcellulose.

Also disclosed herein are methods of use of the disclosed oral care compositions to prevent, treat, and/or mitigate tooth and/or gum sensitivity in the oral cavity.

### BRIEF DESCRIPTION OF THE DRAWINGS

FIG. 1 shows in schematic form an experimental apparatus to maintain calcium ion at constant composition while measuring calcium consumption resulting from calcium oxalate crystal growth in a stirred reaction solution or slurry

FIG. 2A shows a theoretical example of a plot of calcium ion concentration vs. time as small deviations in calcium concentration are quickly reversed to restore constant composition.

FIG. 2B a theoretical example of a plot of calcium ion consumption resulting from the initiation of calcium oxalate crystal growth upon addition of nucleating crystals to a metastable calcium oxalate solution.

FIG. 3 shows a theoretical comparison of plots of calcium ion consumption resulting from calcium oxalate crystal growth, one in which a crystal growth inhibitor is introduced during the crystal growth phase of the experiment. The sharp change in slope of the plot associated with the crystal growth inhibitor represents the quantitative impact of the inhibitor on crystal growth.

FIG. 4 shows an experimental example of plots of calcium consumption resulting from calcium oxalate crystal growth in which carboxymethyl cellulose was added to the stirred reaction slurry at approximately 6 minutes.

FIG. 5 shows an experimental example of plots of calcium consumption resulting from calcium oxalate crystal growth in which two potential crystal growth inhibitors were added to the stirred reaction slurry at approximately 6 minutes.

FIG. 6 is a schematic of a surface-plasmon based experimental apparatus used to directly measure the effect of solution and inhibitor composition on the growth of calcium oxalate crystals bound to a sensor surface.

FIG. 7 is a closeup schematic of an optical apparatus used to measure calcium oxalate crystal growth shown in FIG. 6. (liquid cell omitted for clarity)

FIG. 8 shows an experimental example of a plot of time vs. calcium oxalate crystal growth atop a layer of “seed” crystals attached to the sensor surface. In this case, a metastable solution of calcium oxalate was introduced to the seed crystals at time zero, which initiated crystal growth as measured by the optical sensor. An aliquot of carboxymethylcellulose was introduced at a run time of approximately 13 minutes.

FIG. 9 is a plot showing quantitative inhibition vs. different concentrations of carboxymethyl cellulose in the diluted test solution. For reference, concentrations of carboxymethyl cellulose in the neat product are also shown.

### DETAILED DESCRIPTION OF THE INVENTION

The present invention is directed to oral care compositions that can crystalize material into dentin tubules to reduce tooth sensitivity in the oral cavity of an affected individual. Oxalic acid, and salts thereof, is known to provide sensitivity benefit through partial or full occlusion of dentin tubules through the precipitation and/or crystallization of oxalate salts, such as calcium oxalate.

Without wishing to be bound by theory, it has been found that certain common oral care components can inhibit the crystallization of oxalate salts into dentin tubules. Without wishing to be bound by theory, it has been found that cellulose and cellulose derivatives, such as carboxymethylcellulose can inhibit the crystallization of oxalate salts into dentin tubules. Thus, the disclosed oral care compositions include oxalic acid, or salts thereof, and less than 1%, by weight of the oral care composition, of cellulose derivatives, such as carboxymethylcellulose.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen’ or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise oxalic acid and less than 1%, by weight of the oral care composition, of a cellulose derivative. Additionally, the oral care compositions can comprise other optional ingredients, as described below. The section headers below are provided for convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

### Oxalic Acid

The oral care composition comprises oxalic acid. The oxalic acid can comprise suitable salts of oxalic acid, such as, for example, monoalkali metal oxalate, dialkali metal oxalate, monopotassium monohydrogen oxalate, dipotassium oxalate, monosodium monohydrogen oxalate, disodium oxalate, monopotassium trihydrogen dioxylate, monosodium trihydrogen dioxylate, titanium oxalate, and/or other metal salts of oxalate. The oxalic acid can also include hydrates of the oxalic acid and/or a hydrate of a salt of the oxalic acid.

The oral care composition can comprise from about 0.01% to about 10%, from about 0.1% to about 15%, from about 0.1% to about 5%, or from about 0.0001 to about 25%, of oxalic acid.

### Thickening Agent

The oral care composition can comprise one or more thickening agents. Thickening agents can be useful in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, and/or silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can be substantially free of, essentially free of, or free of a cellulose derivative, such as carboxymethylcellulose. The oral care composition can comprise less than about 1%, less than about 0.5%, or less than about 0.25%, by weight of the oral care composition, of a cellulose derivative, such as carboxymethylcellulose. While not wishing to be bound by theory, it is believed that cellulose derivatives, such as carboxymethylcellulose, can prevent and/or slow the crystallization of oxalic acid within dentin tubules.

The oral care composition can comprise from 0.01% to about 15%, from 0.1% to about 10%, from about 0.2% to about 5%, or from about 0.5% to about 2% of one or more thickening agents.

### pH Buffering Agent

The oral care composition can comprise pH buffering agent, which can adjust the pH, but also provide buffering capacity. The pH buffering agent can have a pKa of from about from about 4 to about 6.5, from about 4.5 to about 6, from about 5 to about 6, or from about 4 to about 7.

The pH buffering agent can comprise monocarboxylic acid, dicarboxylic acid, tricarboxylic acid, tetracarboxylic acid, or combinations thereof.

The monocarboxylic acid can comprise a single carboxylic acid functional group. Suitable compounds can include compounds with the formula R—COOH, wherein R is any organic structure. Suitable monocarboxylic acids can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic

The pH buffering agent can comprise dicarboxylic acid. The dicarboxylic acid comprises a compound with two carboxylic acid functional groups. The dicarboxylic acid can comprise a compound or salt thereof defined by Formula I-A, Formula I-B, and/or Formula I-C.

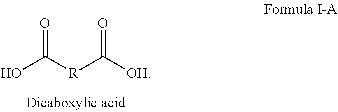

R can be alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

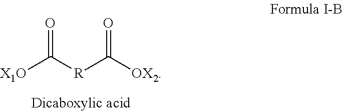

R can be alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

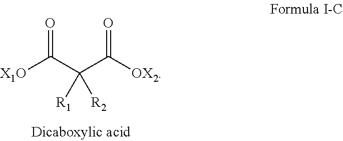

R₁ can be alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

The dicarboxylic acid can be added to a formulation as a neutral acid (as shown in Formula I-A) or as a dicarboxylate monosalt (where one of the carboxylic acid functional groups is a salt and the other is neutral), a dicarboxylate disalt (where both of the carboxylic acid functional groups are salts), or combinations thereof. Additionally, as is well known to a person of ordinary skill in the art, whether or not that one or both of the carboxylic acid functional groups of the dicarboxylic acid are neutral or charged in solution, can be influenced by the pH of the solution. For example, a neutral dicarboxylic acid can be added to an aqueous solution and one or two protons from the two carboxylic acid functional groups can be removed if the pH is lower than the pKa of the carboxylic acid functional group, as shown below in Formula I-D.

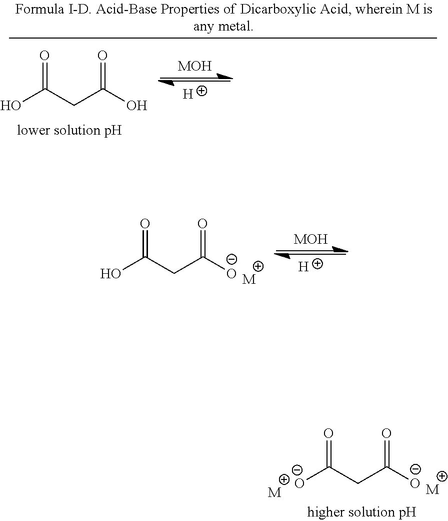

The dicarboxylic acid can comprise malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, dihydroxymalonic acid, fumaric acid, terephthalic acid, glutaric acid, salts thereof, or combinations thereof. The dicarboxylic acid can comprise suitable salts of dicarboxylic acid, such as, for example, when the dicarboxylic acid includes a salt of oxalic acid: monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid. Suitable dicarboxylic acid compounds include dicarboxylic acids described by Formula I-A, wherein R is null, comprises a methylene or ethylene with one or two substitutions, and/or an acetyl group.

Other suitable pH buffering agents include adipic acid, glutaric acid, succinic acid, malonic acid, glutamic acid, ascorbic acid, citric acid, and/or combinations thereof.

The oral care composition can comprise from about 0.01% to about 10%, from about 0.1% to about 15%, from about 1% to about 5%, or from about 0.0001 to about 25%, of pH buffering agent.

pH

The pH of the oral care compositions as described herein can be from about 4 to about 7, from about 4 to about 6.5, from about 4.5 to about 6, from about 4.5 to about 5.5, or from about 4 to about 5.5. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

The pH of the oral care compositions as described herein have a preferred pH of below about 7 or below about 6 due to the pKa of oxalic acid and the pH buffering agent. While not wishing to be bound by theory, it is believed that oxalic acid displays unique behavior when the pH is below about 7 or below about 6, but surfaces in the oral cavity can also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential to damage teeth by acid dissolution is greatly increased. Consequently, the oral care compositions comprising oxalic acid, as described herein, preferably have a pH from about 4 to about 7, from about 4 to about 6, from about 4.5 to about 6.5, from about 4.5 to about 5.5,or from about 4 to about 5.5 to minimize metal hydroxide/metal oxide formation and any damage to oral hard tissues (enamel, dentin, and cementum).

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

The oral care composition of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, or from about 0.3% to about 0.6%, by weight of the dentifrice composition. Alternatively, the oral care composition can be essentially free of, substantially free of, or free of zinc.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

### Abrasive

The oral care composition of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

### Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula II, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

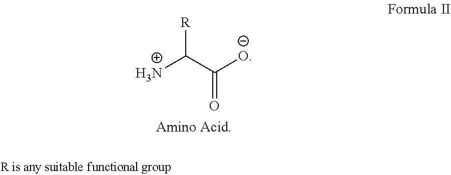

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, or mixtures thereof.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions for the delivery of the oxalic acid include emulsion compositions, such as the emulsion compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, such as the leave-on oral care compositions of U.S. patent application Ser. No. 16/899,834, which is herein incorporated by reference in its entirety, jammed emulsions, such as the jammed oil-in-water emulsion compositions of U.S. Pat. No. 10,780,032, which is herein incorporated by reference in its entirety, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Oral Care Regimen

The oxalic acid can be delivered in the same composition as tin and/or fluoride or the oxalic acid can be delivered in a separate composition. For example, a first composition can comprise tin and/or fluoride and a second composition can comprise oxalic acid and pH buffering agent. The first and second composition can be delivered simultaneously, such as in a dual-phase composition or sequentially from discrete compositions.

An oral care kit can include the first composition comprising tin and/or fluoride and the second composition comprising oxalic acid and pH buffering agent. The oral care kit can also include instructions directing a user to apply the first composition to an oral cavity of the user followed by applying the second composition to the oral cavity of the user. The first composition can be expectorated prior to the application of the second composition or the second composition can be applied prior to the expectoration of the first composition from the oral cavity.

The entire oral care regimen can have a duration of from one minute to about three minutes with each application step having a duration of from about 30 seconds to about 2 minutes or about 1 minute.

The components can be delivered to the oral cavity simultaneously or sequentially. The simplest case is simultaneous, continuous delivery of equal amounts of the two components or a constant ratio of the components during a single oral care session. The two components may be provided separately, such as in a dual-phase composition in two separate compositions, and then delivered simultaneously to the oral cavity. Brushing duration is sufficiently short so that the components will not be inactivated. Another use for simultaneous, continuous delivery is systems that include two components that react relatively slowly, and that will remain in the oral cavity after brushing to be absorbed by the teeth and or gums.

In the case of sequential delivery, both components may be delivered during a single oral care session, e.g., a single brushing session or other single treatment session (single use, start to finish, by a particular user, typically about 0.1 to 5 minutes), or alternatively the components may be delivered individually over multiple oral care sessions. Many combinations are possible, for example delivery of both components during a first oral care session and delivery of only one of the components during a second oral care session.

Sequential delivery during a single oral care session may take various forms. In one case, two components are delivered in alternation, as either a few relatively long duration cycles during brushing (A B A B), or many rapid-fire alternations (A B A B A B A B A B. . . . A B).

In another case, two or more components are delivered one after the other during a single oral care session, with no subsequent alternating delivery in that oral care session (A followed by B). For example, a first composition comprising fluoride and/or tin can be delivered initially, to initiate brushing and provide cleansing, followed by a second composition comprising oxalic acid.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

TABLE 1
Example Compositions

| | Ex. 1 | Ex. 2 | Ex. 3 | Ex. 4 | Ex. 5 | Ex. 6 |
|---|---|---|---|---|---|---|
| | (wt %) | (wt %) | (wt %) | (wt %) % | (wt %) % | (wt %) |
| Sorbitol | 62.000000 | 64.000000 | 64.000000 | 64.000000 | 64.000000 | 45.0000 |
| Water | 8.1450 | 8.5850 | 7.3700 | 6.2170 | 7.2700 | 19.1091 |
| SnF₂ | — | — | — | — | — | 0.4540 |
| SnCl₂/10% | — | — | — | — | — | 0.5619 |
| silica blend | | | | | | |
| NaF | — | — | — | 0.2430 | — | — |
| Sodium Monofluorophosphate | — | — | 1.1400 | — | 1.1400 | — |
| Sodium Gluconate | — | — | — | 1.0000 | — | 1.3000 |
| NaOH 50% | — | — | — | — | — | 0.1500 |
| Saccharin | 0.4000 | 0.4000 | 0.4000 | 0.4000 | 0.4000 | 0.3500 |
| Sucralose Solution (25%) | — | — | — | — | — | 0.0800 |
| Xanthan Gum | 0.5000 | 0.5000 | 0.5000 | 0.5000 | 0.6000 | 0.8750 |
| Carboxymethylcellulose | 1.0000 | 1.0000 | 1.0000 | 1.0000 | — | — |
| Carrageenan | — | — | — | — | 1.0000 | 1.5000 |
| Citric Acid | 0.2150 | 0.2750 | 0.3500 | 0.7000 | 0.3500 | — |
| Na Citrate | — | — | — | 0.7000 | — | 1.2050 |
| Potassium oxalate | 3.1400 | 3.1400 | 3.1400 | 3.1400 | 3.1400 | 3.1400 |
| monohydrate | | | | | | |
| TiO₂ | 0.5000 | 0.5000 | 0.5000 | 0.5000 | 0.5000 | 0.5000 |
| Thickening Silica (Z165) | 3.0000 | 0.5000 | 0.5000 | 0.5000 | 0.5000 | — |
| Abrasive Silica (Z119) | 15.0000 | 15.0000 | 15.0000 | 15.0000 | 15.0000 | 17.5000 |
| SLS Solution (28%) | 5.0000 | 5.0000 | 5.0000 | 5.0000 | 5.0000 | 7.0000 |
| Flavor | 1.1000 | 1.1000 | 1.1000 | 1.1000 | 1.1000 | 1.2750 |

TABLE 1 includes various example oral care compositions. Ex. 1 included potassium oxalate monohydrate and citric acid (pH buffering agent). Ex. 2 included potassium oxalate monohydrate and citric acid (pH buffering agent). Ex. 3 included potassium oxalate monohydrate, citric acid (pH buffering agent), and sodium monofluorophosphate. Ex. 4 included potassium oxalate monohydrate, citric acid (pH buffering agent), sodium citrate (pH buffering agent), and sodium fluoride. Ex. 5 included potassium oxalate monohydrate, citric acid (pH buffering agent), and sodium monofluorophosphate. Ex. 6 potassium oxalate monohydrate, sodium citrate (pH buffering agent), and stannous fluoride.

### Quantitative In Vitro Models of Calcium Oxalate Crystal Growth

To evaluate the degree to which product excipients inhibit calcium oxalate crystal growth, two independent models were employed to quantitatively compare the degree of crystal growth in the presence and absence of individual ingredients. These models were (1) a constant composition model, and (2) a direct surface growth model.

### Constant Composition Model

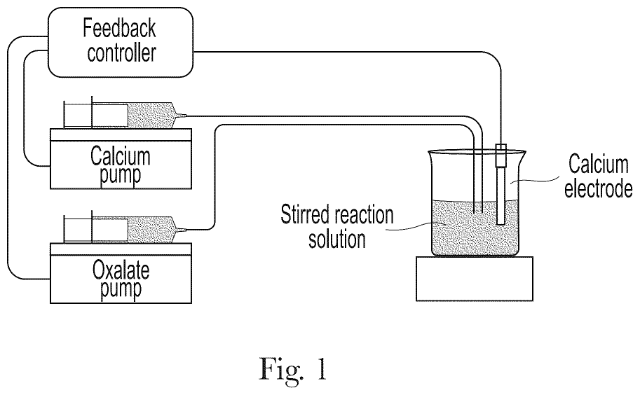

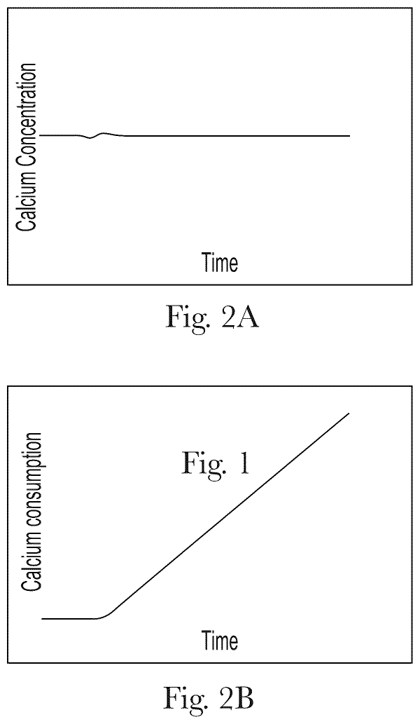

A constant composition model was employed to quantitatively compare the rate of crystal growth in the presence and absence of each ingredient in an oxalate-containing oral care product. This model was adapted from a similar model described by Shechan and Nancollas (Invest Urol, 1980;17(6):446-50), and involves the components shown schematically in FIG. 1. Syringe pumps (Harvard Apparatus model 70-2219) equipped with a 50 ml syringe (Hamilton Gastight Syringe Model 1050 TLL) were used to separately deliver equal volumes of calcium-and oxalate-containing solutions to a stirrer reaction vessel to maintain a state of constant saturation. A feedback controller provided by a commercial software package (National Instruments LabView) controlled the instantaneous pump flow rate calculated from real-time calcium concentrations provided by a meter (Oakton model pH 450) equipped with a calcium ion-selective electrode (ISE) from the Cole-Parmer Instrument Company (p/n EW-27504-06). FIG. 2A is a hypothetical illustration of a plot of calcium ion concentration vs. time as small deviations in calcium concentration are quickly reversed by the feedback controller system to restore and maintain constant composition.

Seed crystals of calcium oxalate dihydrate were prepared according to an adaptation of the procedure published by Doherty et al (Cryst Res Technol. 1994;29(4):517-524), in which 196 ml of an aqueous solution of 4.1 mM sodium oxalate was quickly added to a rapidly stirring solution comprised of 26.1 mM calcium chloride and 16.8 mM sodium citrate. Crystals were collected on a 0.22 um filter, washed 2× in ethanol, and dried before using. Crystal growth, hypothetically represented by steady calcium consumption as shown in FIG. 2B, was initiated by introduction of a 3 ml slurry (seed slurry) of calcium oxalate crystals. The calcium oxalate seed slurry was prepared by rapidly combining 3 ml of purified water to 20 mg calcium oxalate seed crystals for 10 seconds immediately prior to introduction to the reaction solution.

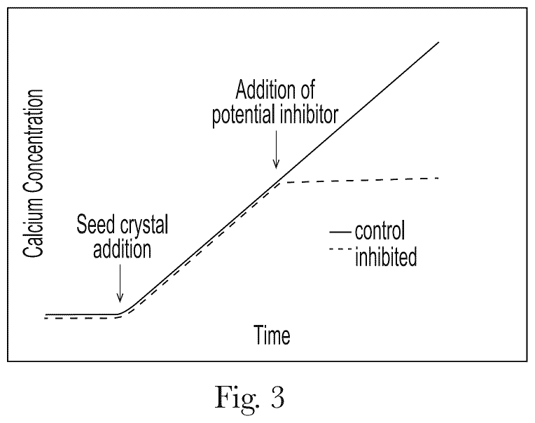

Evaluation of individual compounds or mixtures for detrimental impact on product performance was determined by measuring change to the rate of crystal growth when the compound or mixture was injected into the solution during data collection. Quantitively, the degree to which agents may inhibit crystal growth is expressed in Equation 1,

$$\% I = 100 - 100 ( \frac{{R}_{i}}{{R}_{o}} ) \qquad ( 1 )$$

where % I is the percent inhibition observed, Ro is the native crystal growth rate, measured by the slope in the crystal growth curve prior to addition of the inhibitor, and Ri is the crystal growth rate or slope in the presence of the inhibitor. Thus, a percent inhibition of 90% indicates that crystal growth in the presence of an inhibitor is slowed by a factor of 10 relative to the growth rate in the absence of inhibitors. FIG. 3 is a hypothetical comparison of the crystal growth rate in an inhibitor-free (control) test solution vs. the growth rate in a solution where a substantial crystal growth inhibitor is introduced.

Following the example of Schechan and Nancollas, calcium and oxalate concentrations in test solutions were chosen to exceed the solubility product constant for calcium oxalate, but sufficiently low to ensure that heterogeneous crystal growth occurred primarily on the surface of the seed crystals rather than via spontaneous homogeneous precipitation in solution. This concentration window represents a substantial dilution of the neat product, and is believed to be physiologically relevant, given that (1) substantial dilution of oxalate ion is expected as oxalate ion diffuses from the saliva into dentinal tubuli against the convective flow of pulpal fluid, and (2) the morphology of the relatively large calcium oxalate crystals observed in dentinal tubuli following product application is consistent with heterogeneous precipitation of calcium oxalate (see Hare TC et al, Compend Contin Educ Dent. 2016;37(spec iss 1):13-20). The concentration of potential inhibitors, e.g. sodium carboxylmethylcellulose (CMC), was selected to match the oxalate/inhibitor concentration ratio of 1.5 (oxalate anion/sodium CMC) found in a typical product formulation, see “Ex. 1” 1% CMC composition in Table 1. Table 2 lists dilution factors and oxalate and sodium CMC concentrations in diluted test solutions in the constant composition and direct surface growth models.

TABLE 2
Oxalate and CMC concentrations in diluted test solutions

| | | oxalate | | | |
|---|---|---|---|---|---|
| | | anion/ | | sodium | sodium |
| | dilution | CMC | oxalate | CMC | CMC |
| Model | factor | ratio | (mM) | (ppm) | (% neat)* |
| Constant | 467 | 1.5 | 0.365 | 21 | 1 |
| composition | | | | | |
| Direct | 341 | 150.0 | 0.5 | 0.3 | 0.01 |
| (SPR-based) | | | | | |
| Direct | 341 | 15.0 | 0.5 | 3 | 0.1 |
| (SPR-based) | | | | | |
| Direct | 341 | 1.5 | 0.5 | 30 | 1 |
| (SPR-based) | | | | | |
| Direct | 341 | 0.18 | 0.5 | 240 | 8 |
| (SPR-based) | | | | | |

*Weight % equivalent in neat product

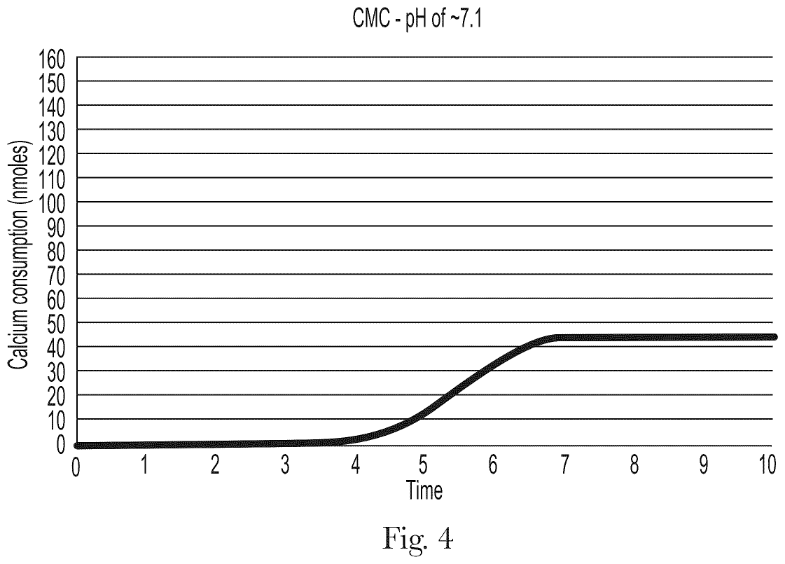

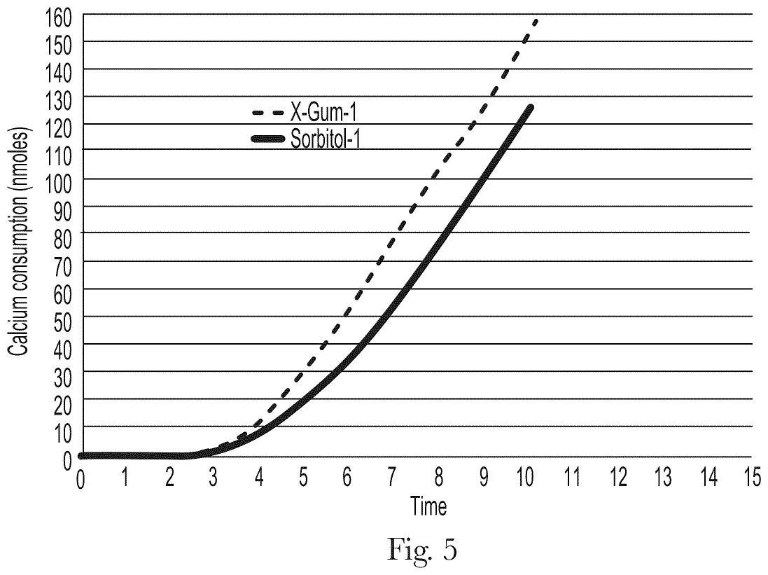

FIG. 4 shows a plot of calcium consumption resulting from calcium oxalate crystal growth in which CMC was added to the stirred reaction slurry at approximately 6 minutes. In this case, calcium and oxalate concentrations were maintained at 0.131 and 0.365 mM, respectively, and a small volume of relatively concentrated CMC was added to achieve a concentration of 23 ppm CMC in solution. The measured % I in this case was 98.9%, which indicates that CMC is a substantial inhibitor of calcium oxalate crystal growth under these conditions, slowing the rate of growth by nearly two orders of magnitude. In contrast, FIG. 5 shows an experimental example of plots of calcium consumption resulting from calcium oxalate crystal growth in which two potential crystal growth inhibitors, xanthan gum and sorbitol, did not significantly change the slope of the crystal growth plot when added to the reaction solution, and were thus shown not to be significant crystal growth inhibitors.

### Direct Growth Model Using Surface Plasmon Resonance

### Utility of Directly Tracking Crystal Growth

Indirect measures of crystal growth, e.g. monitoring loss of calcium from solution via ISE via constant-composition, can be challenging if chelating agents are present in solution. While not wishing to be being bound by theory, it is believed that it can be difficult to distinguish between calcium consumption resulting from crystal growth vs. reduced calcium activity resulting from sequestration. Further compounding this challenge is the fact that some crystal growth inhibitors form relatively strong complexes with calcium ion. Directly tracking crystal growth in real time can mitigate the challenge of distinguishing between various calcium sinks and additionally provides independent confirmation of crystal growth inhibition of key product ingredients.

### Direct Growth Measurements

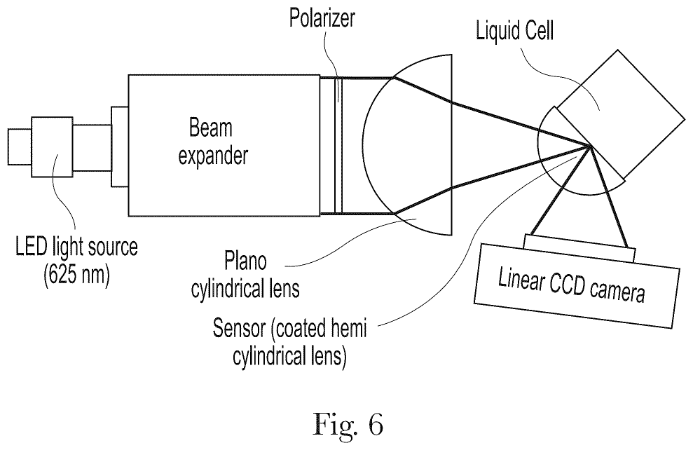

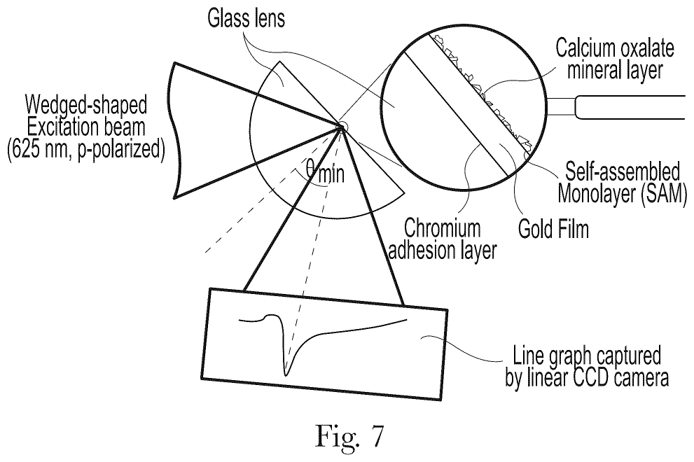

To directly measure calcium oxalate crystal growth, an experimental system based on surface-plasmon resonance spectroscopy was developed and is shown in FIGS. 6 and 7. The sensor was prepared atop a clean hemi-cylindrical lens with high index of refraction (e.g. Schott LaSFN9) by coating the planar surface sequentially with a chromium metal adhesion layer, a gold layer to support the surface plasmon resonance, a carboxylate-based self-assembled monolayer, and finally a salivary or protein pellicle as shown in FIG. 7. Chromium and gold layers were deposited using a conventional sputter coater at 2.5 nm and 47.5 nm, respectively. The self-assembled monolayer (SAM) was deposited by immersing the chromium and gold-covered lens in a 1 mM solution of 11-mercaptoundecanoic acid in ethanol for 30-60 minutes, followed by a rinse in ethanol. The SAM-covered sensor was then stored in 75 mM NaCl until use.

Deposition data was acquired on a linear, 2048-pixel CCD camera at suitable intervals to track the progress of average crystal growth rate. As shown in FIGS. 6 and 7, an internally reflected wedge of p-polarized light from a 632 nm LED was used to excite a surface plasmon resonance in the sensor surface. Labview software was used to identify the minimum position (pixel minimum) in the reflectance spectrum collected on the camera. Because the change in angular position of the reflectivity minimum is sensitive to and approximately linearly related to the change in average thickness of a thin layer atop the sensor, fractional crystal growth rate reductions may be readily approximated from changes in the slope of pixel min vs time plots. Changes in crystal growth rates are more accurately calculated from changes in the slope of crystal layer thicknesses vs. time, where the approximate average thickness of crystal layers is directly calculated from Fresnel's equations (see Electromagnetic Surface Modes, 1982, John Wiley & Sons, pp 143-200. Boardman, A D, Ed.). In this case, the angle associated with each pixel min is calculated from the experimental geometry.

Seed crystals of calcium oxalate were deposited on the pellicle-covered sensor surface by exposure of the SAM-covered surface to a freshly combined solution of calcium and oxalate (0.375 mM each in 150 mM NaCl and 10 mM HEPES buffer) at pH 7 for at least 90 minutes. Once a layer of calcium oxalate seed crystals was established on the sensor surface, the sensor surface was flushed with a calcium oxalate solution at the saturation threshold after which the sensor was placed in a clean cell before performing crystal growth rate measurements.

To obtain data necessary to calculate fractional crystal growth rate reductions caused by potential inhibitors, data was collected during a period of linear crystal growth before and after the test component was introduced. Crystal growth was typically initiated by combining equal volumes of 1.0 mM calcium chloride and 1.0 mM sodium oxalate solutions to achieve a saturated solution at 0.5 mM calcium and 0.5 mM oxalate. Both solutions contained 150 mM NaCl to maintain a constant ionic strength.

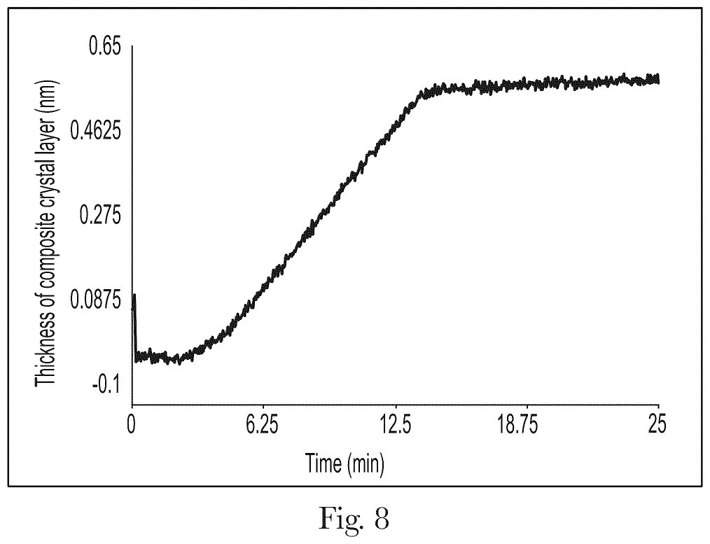

FIG. 8 shows data collected when (1) crystal growth was initiated by combining 40 mL of 1.0 mM calcium chloride and 40 mL of 1.0 mM sodium oxalate over the sensor, followed by the introduction of 470 μL of a 0.5% solution of CMC at 13 minutes. It is evident from the sharp reduction in slope, which corresponds to a measured inhibition value of 96.4%, that sodium carboxymethylcellulose is a substantial crystal growth inhibitor under these conditions.

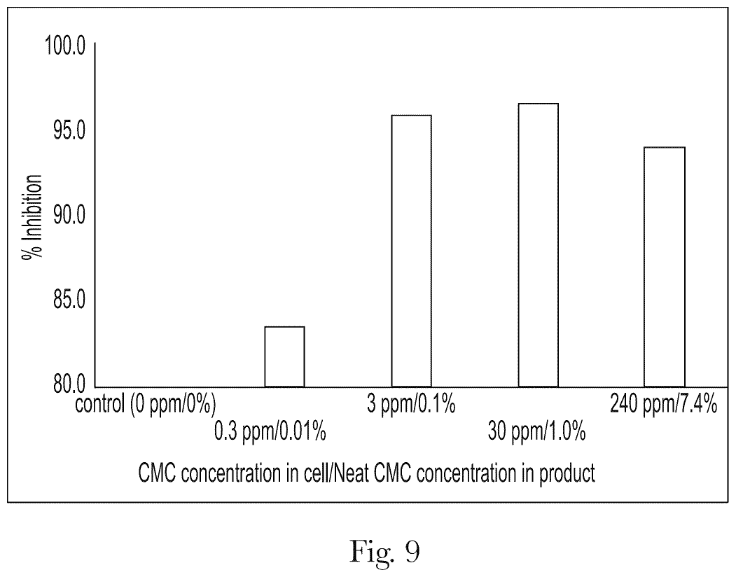

CMC-induced inhibition was observed to be concentration-dependent as shown in FIG. 9. Inhibition increases with carboxymethylcellulose concentration to at least 30 ppm. Although some inhibition was apparent with concentrations as dilute as 30 ppb, this was determined to be likely insignificant in normal usage given that the delay between CMC introduction and observed inhibition was significantly longer than the typical 60 second product application window. For reference, Table 2 lists dilution factors and oxalate/sodium CMC concentration ratios for the dilute test solutions and equivalent neat product formulations shown along the axis of abscissas in FIG. 9.

Cellulose derivatives, such as carboxymethylcellulose, were found to be a significant inhibitor of calcium oxalate crystal growth using two complementary kinetic models. Specifically, concentrations as low as 0.3 ppm resulted in a several-fold decrease in crystal growth rates. Assuming equal dilution of carboxymethylcellulose and oxalate ion in vivo, this corresponds to neat, applied product concentration of roughly 0.01% and above.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

what is claimed is:

1. A dentifrice composition comprising:
(a) about 1% to about 10%, by weight of the composition, of oxalic acid or a salt thereof;
(b) 0% to about 35%, by weight of the composition, of water; and
(c) a thickening agent comprising xanthan gum and carageenan,
wherein the oral care composition is free of cellulose derivative, and
wherein the oral care composition has a pH of about 4 to about 7, and wherein the composition has a percent inhibition (% I) of calcium oxalate crystal growth of less than 90% according to the Constant Composition Model.

2. The dentifrice composition of claim 1, wherein the oral care composition comprises pH buffering agent with a pKa of about 4.5 to about 6.

3. The dentifrice composition of claim 4, wherein the pH buffering agent comprises adipic acid, glutaric acid, succinic acid, malonic acid, glutamic acid, ascorbic acid, citric acid, salts thereof, or combinations thereof.

4. The dentifrice composition of claim 1, wherein the oxalic acid comprises oxalic acid, monosodium monohydrogen oxalate, disodium oxalate, monopotassium monohydrogen oxalate, dipotassium oxalate, calcium oxalate, or combinations thereof.

5. The dentifrice composition of claim 1, wherein the oral care composition comprises fluoride.

6. The dentifrice composition of claim 1, further comprising a metal selected from tin, zinc, copper, or combinations thereof.

7. The dentifrice composition of claim 1, wherein the oral care composition comprises a polyphosphate selected from comprises pyrophosphate, tripolyphosphate, tetrapolyphosphate, hexametaphosphate, or combinations thereof.

8. The dentifrice composition of claim 1, wherein the oral care composition a silica abrasive, a calcium abrasive, or a combination thereof.

9. The dentifrice composition of claim 1, wherein the pH is about 4 to about 6.

10. A method of preventing, treating, or mitigating sensitivity in an oral cavity of a user, the method comprising:
a) directing the user to apply the oral care composition of claim 1 to at least one tooth in the oral cavity for an application period of about 30 seconds to about 2 minutes; and
b) directing the user to expectorate the oral care composition after the application period.

11. The method of claim 10, wherein the oral care composition does not form a film, varnish, or combinations thereof in the oral cavity.

12. A dentifrice composition comprising:
a) about 0.01% to about 10%, by weight of the dentifrice composition, of an oxalic or oxalate compound selected from oxalic acid, monosodium monohydrogen oxalate, disodium oxalate, monopotassium monohydrogen oxalate, dipotassium oxalate, calcium oxalate, and combinations thereof;
b) about 20% to about 80%, by weight of the dentifrice composition, of sorbitol;
c) sodium fluoride;
d) sodium gluconate; and
e) about 0.6% to about 1% xanthan gum and about 1% to about 1.5% carrageenan, based on the weight of the dentifrice composition,
wherein the dentifrice composition is free of carboxymethylcellulose, and wherein the composition has a % I of calcium oxalate crystal growth of less than 90% according to the Constant Composition Model.

13. The dentifrice composition of claim 1, wherein the xanthan gum is present at about 0.6% to about 1%, based on the weight of the dentifrice composition.

14. The dentifrice composition of claim 1, wherein the carrageenan is present at about 1% to about 1.5%, based on the weight of the dentifrice composition.
